SEQUENCE LISTING (16 sequences)

SEQ ID NO: 1 - DNA, 62 bp, Artificial Sequence
  Basic Sequence 1
aatgatacggcgaccaccgagatctacacacactctttccctacacgacgctcttccgatct

SEQ ID NO: 2 - DNA, 58 bp, Artificial Sequence
  Basic Sequence 2
agatcggaagagcacacgtctgaactccagtcacatctcgtatgccgtcttctgcttg

SEQ ID NO: 3 - DNA, 58 bp, Artificial Sequence
  Basic Sequence 3
ctgtctcttatacacatctccgagcccacgagacatctcgtatgccgtcttctgcttg

SEQ ID NO: 4 - DNA, 62 bp, Artificial Sequence
  Basic Sequence 4
ctgtctcttatacacatctgacgctgccgacgagtgtagatctcggtggtcgccgtatcatt

SEQ ID NO: 5 - DNA, 63 bp, Artificial Sequence
  Oligonucleotide 1 in Example 1
  misc_feature: 3′-3SpC3 modification at 3′-end
  misc_feature (8)..(8): LNA-C at position 8
  misc_feature (11)..(11): LNA-C at position 11
  misc_feature (14)..(14): LNA-C at position 14
  misc_feature (17)..(17): LNA-C at position 17
  misc_feature (24)..(24): LNA-C at position 24
  misc_feature (30)..(30): n is a hexaethylene glycol phosphodiester bond structure
  misc_feature (32)..(32): LNA-C at position 32
  misc_feature (34)..(34): LNA-C at position 34
  misc_feature (36)..(36): LNA-C at position 36
  misc_feature (40)..(40): LNA-C at position 40
  misc_feature (42)..(42): LNA-C at position 42
  misc_feature (45)..(45): LNA-C at position 45
  misc_feature (47)..(47): LNA-C at position 47
  misc_feature (50)..(50): LNA-C at position 50
  misc_feature (52)..(52): LNA-C at position 52
  misc_feature (54)..(54): LNA-C at position 54
  misc_feature (58)..(58): LNA-C at position 58
aatgatacggcgaccaccgagatctacacnacactctttccctacacgacgctcttccgatct

SEQ ID NO: 6 - DNA, 59 bp, Artificial Sequence
  Oligonucleotide 2 in Example 1
  misc_feature: 3′-3SpC3 modification at 3′-end
  misc_feature (5)..(5): LNA-C at position 5
  misc_feature (13)..(13): LNA-C at position 13
  misc_feature (15)..(15): LNA-C at position 15
  misc_feature (17)..(17): LNA-C at position 17
  misc_feature (20)..(20): LNA-C at position 20
  misc_feature (25)..(25): LNA-C at position 25
  misc_feature (27)..(27): LNA-C at position 27
  misc_feature (28)..(28): LNA-C at position 28
  misc_feature (32)..(32): LNA-C at position 32
  misc_feature (35)..(35): n is a hexaethylene glycol phosphodiester bond structure
  misc_feature (38)..(38): LNA-C at position 38
  misc_feature (40)..(40): LNA-C at position 40
  misc_feature (46)..(46): LNA-C at position 46
  misc_feature (47)..(47): LNA-C at position 47
  misc_feature (50)..(50): LNA-C at position 50
  misc_feature (53)..(53): LNA-C at position 53
  misc_feature (56)..(56): LNA-C at position 56
agatcggaagagcacacgtctgaactccagtcacnatctcgtatgccgtcttctgcttg

SEQ ID NO: 7 - DNA, 70 bp, Artificial Sequence
  Oligonucleotide 1 in Example 2
  misc_feature: 3′-3SpC3 modification at 3′-end
  misc_feature (8)..(8): LNA-C at position 8
  misc_feature (11)..(11): LNA-C at position 11
  misc_feature (14)..(14): LNA-C at position 14
  misc_feature (17)..(17): LNA-C at position 17
  misc_feature (24)..(24): LNA-C at position 24
  misc_feature (30)..(37): n is an ethylene glycol phosphodiester bond structure
  misc_feature (39)..(39): LNA-C at position 39
  misc_feature (41)..(41): LNA-C at position 41
  misc_feature (43)..(43): LNA-C at position 43
  misc_feature (47)..(47): LNA-C at position 47
  misc_feature (49)..(49): LNA-C at position 49
  misc_feature (52)..(52): LNA-C at position 52
  misc_feature (54)..(54): LNA-C at position 54
  misc_feature (57)..(57): LNA-C at position 57
  misc_feature (59)..(59): LNA-C at position 59
  misc_feature (61)..(61): LNA-C at position 61
  misc_feature (65)..(65): LNA-C at position 65
aatgatacggcgaccaccgagatctacacnnnnnnnnacactctttccctacacgacgctcttccgatct

SEQ ID NO: 8 - DNA, 66 bp, Artificial Sequence
  Oligonucleotide 2 in Example 2
  misc_feature: 3′-3SpC3 modification at 3′-end
  misc_feature (5)..(5): LNA-C at position 5
  misc_feature (13)..(13): LNA-C at position 13
  misc_feature (15)..(15): LNA-C at position 15
  misc_feature (17)..(17): LNA-C at position 17
  misc_feature (20)..(20): LNA-C at position 20
  misc_feature (25)..(25): LNA-C at position 25
  misc_feature (27)..(27): LNA-C at position 27
  misc_feature (28)..(28): LNA-C at position 28
  misc_feature (32)..(32): LNA-C at position 32
  misc_feature (35)..(42): n is an ethylene glycol phosphodiester bond structure
  misc_feature (45)..(45): LNA-C at position 45
  misc_feature (47)..(47): LNA-C at position 47
  misc_feature (53)..(53): LNA-C at position 53
  misc_feature (54)..(54): LNA-C at position 54
  misc_feature (57)..(57): LNA-C at position 57
  misc_feature (60)..(60): LNA-C at position 60
  misc_feature (63)..(63): LNA-C at position 63
agatcggaagagcacacgtctgaactccagtcacnnnnnnnnatctcgtatgccgtcttctgcttg

SEQ ID NO: 9 - DNA, 69 bp, Artificial Sequence
  Oligonucleotide 1 in Example 3
  misc_feature: 3′-3SpC3 modification at 3′-end
  misc_feature (8)..(8): LNA-C at position 8
  misc_feature (11)..(11): LNA-C at position 11
  misc_feature (14)..(14): LNA-C at position 14
  misc_feature (17)..(17): LNA-C at position 17
  misc_feature (24)..(24): LNA-C at position 24
  misc_feature (30)..(36): n is a propylene glycol phosphodiester bond structure
  misc_feature (38)..(38): LNA-C at position 38
  misc_feature (40)..(40): LNA-C at position 40
  misc_feature (42)..(42): LNA-C at position 42
  misc_feature (46)..(46): LNA-C at position 46
  misc_feature (48)..(48): LNA-C at position 48
  misc_feature (51)..(51): LNA-C at position 51
  misc_feature (53)..(53): LNA-C at position 53
  misc_feature (56)..(56): LNA-C at position 56
  misc_feature (58)..(58): LNA-C at position 58
  misc_feature (60)..(60): LNA-C at position 60
  misc_feature (64)..(64): LNA-C at position 64
aatgatacggcgaccaccgagatctacacnnnnnnnacactctttccctacacgacgctcttccgatct

SEQ ID NO: 10 - DNA, 65 bp, Artificial Sequence
  Oligonucleotide 2 in Example 3
  misc_feature: 3′-3SpC3 modification at 3′-end
  misc_feature (5)..(5): LNA-C at position 5
  misc_feature (13)..(13): LNA-C at position 13
  misc_feature (15)..(15): LNA-C at position 15
  misc_feature (17)..(17): LNA-C at position 17
  misc_feature (20)..(20): LNA-C at position 20
  misc_feature (25)..(25): LNA-C at position 25
  misc_feature (27)..(27): LNA-C at position 27
  misc_feature (28)..(28): LNA-C at position 28
  misc_feature (32)..(32): LNA-C at position 32
  misc_feature (35)..(41): n is a propylene glycol phosphodiester bond structure
  misc_feature (44)..(44): LNA-C at position 44
  misc_feature (46)..(46): LNA-C at position 46
  misc_feature (52)..(52): LNA-C at position 52
  misc_feature (53)..(53): LNA-C at position 53
  misc_feature (56)..(56): LNA-C at position 56
  misc_feature (59)..(59): LNA-C at position 59
  misc_feature (62)..(62): LNA-C at position 62
agatcggaagagcacacgtctgaactccagtcacnnnnnnnatctcgtatgccgtcttctgcttg

SEQ ID NO: 11 - DNA, 66 bp, Artificial Sequence
  Oligonucleotide 1 in Example 4
  misc_feature: 3′-3SpC3 modification at 3′-end
  misc_feature (5)..(5): LNA-C at position 5
  misc_feature (7)..(7): LNA-C at position 7
  misc_feature (13)..(13): LNA-C at position 13
  misc_feature (15)..(15): LNA-C at position 15
  misc_feature (18)..(18): LNA-C at position 18
  misc_feature (21)..(21): LNA-C at position 21
  misc_feature (25)..(25): LNA-C at position 25
  misc_feature (27)..(27): LNA-C at position 27
  misc_feature (29)..(29): LNA-C at position 29
  misc_feature (34)..(34): LNA-C at position 34
  misc_feature (35)..(42): n is an ethylene glycol phosphodiester bond structure
  misc_feature (45)..(45): LNA-C at position 45
  misc_feature (47)..(47): LNA-C at position 47
  misc_feature (54)..(54): LNA-C at position 54
  misc_feature (57)..(57): LNA-C at position 57
  misc_feature (60)..(60): LNA-C at position 60
  misc_feature (63)..(63): LNA-C at position 63
ctgtctcttatacacatctccgagcccacgagacnnnnnnnnatctcgtatgccgtcttctgcttg

SEQ ID NO: 12 - DNA, 70 bp, Artificial Sequence
  Oligonucleotide 2 in Example 4
  misc_feature: 3′-3SpC3 modification at 3′-end
  misc_feature (5)..(5): LNA-C at position 5
  misc_feature (7)..(7): LNA-C at position 7
  misc_feature (13)..(13): LNA-C at position 13
  misc_feature (15)..(15): LNA-C at position 15
  misc_feature (18)..(18): LNA-C at position 18
  misc_feature (22)..(22): LNA-C at position 22
  misc_feature (24)..(24): LNA-C at position 24
  misc_feature (28)..(28): LNA-C at position 28
  misc_feature (31)..(31): LNA-C at position 31
  misc_feature (34)..(41): n is an ethylene glycol phosphodiester bond structure
  misc_feature (50)..(50): LNA-C at position 50
  misc_feature (52)..(52): LNA-C at position 52
  misc_feature (59)..(59): LNA-C at position 59
  misc_feature (61)..(61): LNA-C at position 61
  misc_feature (67)..(67): LNA-C at position 67
ctgtctcttatacacatctgacgctgccgacgannnnnnnngtgtagatctcggtggtcgccgtatcatt

SEQ ID NO: 13 - DNA, 66 bp, Artificial Sequence
  Oligonucleotide 1 in Example 5
  misc_feature: 3′-3SpC3 modification at 3′-end
  misc_feature (1)..(1): 2′-methoxy modification and phosphorothioate modification at position 1
  misc_feature (2)..(2): 2′-methoxy modification and phosphorothioate modification at position 2
  misc_feature (5)..(5): LNA-C at position 5
  misc_feature (7)..(7): LNA-C at position 7
  misc_feature (13)..(13): LNA-C at position 13
  misc_feature (15)..(15): LNA-C at position 15
  misc_feature (18)..(18): LNA-C at position 18
  misc_feature (21)..(21): LNA-C at position 21
  misc_feature (25)..(25): LNA-C at position 25
  misc_feature (27)..(27): LNA-C at position 27
  misc_feature (29)..(29): LNA-C at position 29
  misc_feature (34)..(34): LNA-C at position 34
  misc_feature (35)..(42): n is an ethylene glycol phosphodiester bond structure
  misc_feature (45)..(45): LNA-C at position 45
  misc_feature (47)..(47): LNA-C at position 47
  misc_feature (54)..(54): LNA-C at position 54
  misc_feature (57)..(57): LNA-C at position 57
  misc_feature (60)..(60): LNA-C at position 60
  misc_feature (63)..(63): LNA-C at position 63
  misc_feature (64)..(64): Phosphorothioate modification at position 64
  misc_feature (65)..(65): 2′-methoxy modification and phosphorothioate modification at position 65
  misc_feature (66)..(66): 2′-methoxy modification at position 66
cugtctcttatacacatctccgagcccacgagacnnnnnnnnatctcgtatgccgtcttctgctug

SEQ ID NO: 14 - DNA, 70 bp, Artificial Sequence
  Oligonucleotide 2 in Example 5
  misc_feature: 3′-3SpC3 modification at 3′-end
  misc_feature (1)..(1): 2′-methoxy modification and phosphorothioate modification at position 1
  misc_feature (2)..(2): 2′-methoxy modification and phosphorothioate modification at position 2
  misc_feature (5)..(5): LNA-C at position 5
  misc_feature (7)..(7): LNA-C at position 7
  misc_feature (13)..(13): LNA-C at position 13
  misc_feature (15)..(15): LNA-C at position 15
  misc_feature (18)..(18): LNA-C at position 18
  misc_feature (22)..(22): LNA-C at position 22
  misc_feature (24)..(24): LNA-C at position 24
  misc_feature (28)..(28): LNA-C at position 28
  misc_feature (31)..(31): LNA-C at position 31
  misc_feature (34)..(41): n is an ethylene glycol phosphodiester bond structure
  misc_feature (45)..(45): LNA-T at position 45
  misc_feature (50)..(50): LNA-C at position 50
  misc_feature (52)..(52): LNA-C at position 52
  misc_feature (55)..(55): LNA-T at position 55
  misc_feature (59)..(59): LNA-C at position 59
  misc_feature (61)..(61): LNA-C at position 61
  misc_feature (67)..(67): LNA-C at position 67
  misc_feature (68)..(68): Phosphorothioate modification at position 68
  misc_feature (69)..(69): 2′-methoxy modification and phosphorothioate modification at position 69
  misc_feature (70)..(70): 2′-methoxy modification at position 70
cugtctcttatacacatctgacgctgccgacgannnnnnnngtgtagatctcggtggtcgccgtatcauu

SEQ ID NO: 15 - DNA, 65 bp, Artificial Sequence
  Oligonucleotide 1 in Example 6
  misc_feature: 3′-3SpC3 modification at 3′-end
  misc_feature (5)..(5): at position 5LNA-C
  misc_feature (7)..(7): LNA-C at position 7
  misc_feature (13)..(13): LNA-C at position 13
  misc_feature (15)..(15): LNA-C at position 15
  misc_feature (18)..(18): LNA-C at position 18
  misc_feature (21)..(21): LNA-C at position 21
  misc_feature (25)..(25): LNA-C at position 25
  misc_feature (27)..(27): LNA-C at position 27
  misc_feature (29)..(29): LNA-C at position 29
  misc_feature (34)..(34): LNA-C at position 34
  misc_feature (35)..(41): n is a propylene glycol phosphodiester bond structure
  misc_feature (44)..(44): LNA-C at position 44
  misc_feature (46)..(46): LNA-C at position 46
  misc_feature (53)..(53): LNA-C at position 53
  misc_feature (56)..(56): LNA-C at position 56
  misc_feature (59)..(59): LNA-C at position 59
  misc_feature (62)..(62): LNA-C at position 62
ctgtctcttatacacatctccgagcccacgagacnnnnnnnatctcgtatgccgtcttctgcttg

SEQ ID NO: 16 - DNA, 69 bp, Artificial Sequence
  Oligonucleotide 2 in Example 6
  misc_feature: 3′-3SpC3 modification at 3′-end
  misc_feature (5)..(5): LNA-C at position 5
  misc_feature (7)..(7): LNA-C at position 7
  misc_feature (13)..(13): LNA-C at position 13
  misc_feature (15)..(15): LNA-C at position 15
  misc_feature (18)..(18): LNA-C at position 18
  misc_feature (22)..(22): LNA-C at position 22
  misc_feature (24)..(24): LNA-C at position 24
  misc_feature (28)..(28): LNA-C at position 28
  misc_feature (31)..(31): LNA-C at position 31
  misc_feature (34)..(40): n is a propylene glycol phosphodiester bond structure
  misc_feature (44)..(44): LNA-T at position 44
  misc_feature (49)..(49): LNA-C at position 49
  misc_feature (51)..(51): LNA-C at position 51
  misc_feature (54)..(54): LNA-T at position 54
  misc_feature (58)..(58): LNA-C at position 58
  misc_feature (60)..(60): LNA-C at position 60
  misc_feature (66)..(66): LNA-C at position 66
ctgtctcttatacacatctgacgctgccgacgannnnnnngtgtagatctcggtggtcgccgtatcatt

(19) United States
(12) Patent Application Publication
(10) Pub. No.: US 2022/0348606 A1
(43) Pub. Date: Nov. 3, 2022

(54) OLIGONUCLEOTIDE CONTAINING BLOCKER

(71) Applicant: Nanjing GenScript Biotech Co., Ltd., Nanjing (CN)

(72) Inventors: Jianpeng Wang, Nanjing (CN); Yu Zhao, Nanjing (CN); Cheng-Hsien Wu, Nanjing (CN)

(73) Assignee: Nanjing GenScript Biotech Co., Ltd., Nanjing (CN)

(21) Appl. No.: 17/617,080

(22) PCT Filed: Jun. 28, 2020
(86) PCT No.: PCT/CN2020/098473

§ 371 Date: Dec. 7, 2021

(30) Foreign Application Priority Data
Jun. 26, 2019 (CN) 201910560841.0

(51) Int. Cl. C07H 21/00; C12Q 1/6874; C12Q 1/686

(52) U.S. Cl. CPC C07H 21/00; C12Q 1/6874; C12Q 1/686

(57) ABSTRACT

An oligonucleotide containing a blocker, relating to the field of target sequence hybridization capture and the design and synthesis of universal blocking oligonucleotides. Applying an oligonucleotide that has a blocking function or a combination thereof not only has a good blocking effect on an linker sequence during the capture of a target sequence in a single sample, but also reduces non-specific capture and improves the capture efficiency. In particular, the oligonucleotide may effectively block linker sequences at both ends of a target sequence in a plurality of samples and improve the target sequence capture efficiency.

### TECHNICAL FIELD

The present application relates to the field of next-generation sequencing, and specifically, to the field of target region hybridization capture and universal blocking oligonucleotide sequence design and synthesis.

### BACKGROUND

Target region hybridization capture is a diagnostic technology based on next-generation sequencing and belongs to the field of personalized medicine and companion diagnostics. The human genome encodes a large amount of genetic information, having 23 pairs of chromosomes and 20,000 to 25,000 genes. A large number of studies have shown that base mutations at specific sites in some genes can be used as biomarkers for the diagnosis of some diseases. Although the cost of whole exome sequencing and even genome sequencing has been significantly reduced with the development of next-generation sequencing technology, if the whole exome sequencing or genome sequencing is used only for detection of a certain type of disease, it will cause serious waste of sequencing throughput and increase of detection costs. To improve sequencing efficiency and reduce detection costs, genes related to a certain type of disease are usually screened in combination, and only these genes are sequenced.

In this case, a key step is to extract and isolate target genes from the genome. The commonly method used in the market is to design primer probes that can target bind to the target gene sequence, so as to isolate the target gene sequence from many heterogeneous gene sequences. The primer probe usually contains biotin modification. After it binds to the target gene sequence, specific purification can be carried out by using magnetic beads coated with streptavidin.

Illumina's next-generation sequencer is widely used in this field. After the genomic DNA is extracted, capture sample preparation will be carried out according to Illumina's library preparation kits. The genomic DNA is usually fragmented, fragments ranging from 300 to 500 bp in length are screened out, and reverse complementary adapter sequences are added at both ends of the fragment for sequencing. In this case, in addition to that the biotin-modified primer probes can bind to the target gene sequence to achieve isolation by magnetic beads, the gene sequence in the non-target region can also be hybridized with complementary adapter sequences at both ends and indirectly isolated by magnetic beads, thereby resulting in non-specific capture. To solve this problem, a large number of oligonucleotide sequences that contain blockers and are reverse complementary to the adapter sequences can be added to the hybridization system. However, when multiple samples are captured simultaneously for target sequences, a universal blocking oligonucleotide sequence cannot be found due to the inconsistent index sequence within the adapter sequence in different samples.

### SUMMARY

To solve the foregoing technical problem, according to an aspect, the present application provides an oligonucleotide containing a blocker introduced into a basic sequence, where the backbone of the blocker contains a carbon-carbon bond, a carbon-oxygen bond, a carbon-nitrogen bond, a nitrogen-oxygen bond, a phosphorus-oxygen bond, or a combination thereof, and the quantity of the carbon-carbon bond, the carbon-oxygen bond, the carbon-nitrogen bond, the nitrogen-oxygen bond, the phosphorus-oxygen bond, or the combination thereof is 10-900.

In some embodiments, the quantity of the carbon-carbon bond, the carbon-oxygen bond, the carbon-nitrogen bond, the nitrogen-oxygen bond, the phosphorus-oxygen bond, or the combination thereof is 20-200.

In some embodiments, the quantity of the carbon-carbon bond, the carbon-oxygen bond, the carbon-nitrogen bond, the nitrogen-oxygen bond, the phosphorus-oxygen bond, or the combination thereof is 20.

In some embodiments, the quantity of the carbon-carbon bond, the carbon-oxygen bond, the carbon-nitrogen bond, the nitrogen-oxygen bond, the phosphorus-oxygen bond, or the combination thereof is 30-54.

In some embodiments, the quantity of the carbon-carbon bond, the carbon-oxygen bond, the carbon-nitrogen bond, the nitrogen—oxygen bond, the phosphorus-oxygen bond, or the combination thereof is 31.

In some embodiments, the quantity of the carbon-carbon bond, the carbon-oxygen bond, the carbon-nitrogen bond, the nitrogen-oxygen bond, the phosphorus-oxygen bond, or the combination thereof is 32.

In some embodiments, the quantity of the carbon-carbon bond, the carbon-oxygen bond, the carbon-nitrogen bond, the nitrogen-oxygen bond, the phosphorus-oxygen bond, or the combination thereof is 33.

In some embodiments, the quantity of the carbon-carbon bond, the carbon-oxygen bond, the carbon-nitrogen bond, the nitrogen-oxygen bond, the phosphorus-oxygen bond, or the combination thereof is 34.

In some embodiments, the quantity of the carbon-carbon bond, the carbon—oxygen bond, the carbon-nitrogen bond, the nitrogen-oxygen bond, the phosphorus-oxygen bond, or the combination thereof is 35.

In some embodiments, the quantity of the carbon-carbon bond, the carbon-oxygen bond, the carbon-nitrogen bond, the nitrogen-oxygen bond, the phosphorus-oxygen bond, or the combination thereof is 36.

In some embodiments, the quantity of the carbon-carbon bond, the carbon-oxygen bond, the carbon-nitrogen bond, the nitrogen-oxygen bond, the phosphorus-oxygen bond, or the combination thereof is 37.

In some embodiments, the quantity of the carbon-carbon bond, the carbon-oxygen bond, the carbon-nitrogen bond, the nitrogen-oxygen bond, the phosphorus-oxygen bond, or the combination thereof is 38.

In some embodiments, the quantity of the carbon-carbon bond, the carbon-oxygen bond, the carbon-nitrogen bond, the nitrogen-oxygen bond, the phosphorus-oxygen bond, or the combination thereof is 39.

In some embodiments, the quantity of the carbon-carbon bond, the carbon-oxygen bond, the carbon-nitrogen bond, the nitrogen-oxygen bond, the phosphorus-oxygen bond, or the combination thereof is 40.

In some embodiments, the quantity of the carbon-carbon bond, the carbon-oxygen bond, the carbon-nitrogen bond, the nitrogen-oxygen bond, the phosphorus-oxygen bond, or the combination thereof is 41.

In some embodiments, the quantity of the carbon-carbon bond, the carbon-oxygen bond, the carbon-nitrogen bond, the nitrogen-oxygen bond, the phosphorus-oxygen bond, or the combination thereof is 42.

In some embodiments, the quantity of the carbon-carbon bond, the carbon-oxygen bond, the carbon-nitrogen bond, the nitrogen-oxygen bond, the phosphorus-oxygen bond, or the combination thereof is 43.

In some embodiments, the quantity of the carbon-carbon bond, the carbon-oxygen bond, the carbon-nitrogen bond, the nitrogen-oxygen bond, the phosphorus-oxygen bond, or the combination thereof is 44.

In some embodiments, the quantity of the carbon-carbon bond, the carbon-oxygen bond, the carbon-nitrogen bond, the nitrogen-oxygen bond, the phosphorus-oxygen bond, or the combination thereof is 45.

In some embodiments, the quantity of the carbon-carbon bond, the carbon-oxygen bond, the carbon-nitrogen bond, the nitrogen-oxygen bond, the phosphorus-oxygen bond, or the combination thereof is 46.

In some embodiments, the quantity of the carbon-carbon bond, the carbon-oxygen bond, the carbon-nitrogen bond, the nitrogen-oxygen bond, the phosphorus-oxygen bond, or the combination thereof is 47.

In some embodiments, the quantity of the carbon-carbon bond, the carbon-oxygen bond, the carbon-nitrogen bond, the nitrogen-oxygen bond, the phosphorus-oxygen bond, or the combination thereof is 48.

In some embodiments, the quantity of the carbon-carbon bond, the carbon-oxygen bond, the carbon-nitrogen bond, the nitrogen-oxygen bond, the phosphorus-oxygen bond, or the combination thereof is 49.

In some embodiments, the quantity of the carbon-carbon bond, the carbon-oxygen bond, the carbon-nitrogen bond, the nitrogen-oxygen bond, the phosphorus-oxygen bond, or the combination thereof is 50.

In some embodiments, the quantity of the carbon-carbon bond, the carbon-oxygen bond, the carbon-nitrogen bond, the nitrogen-oxygen bond, the phosphorus-oxygen bond, or the combination thereof is 51.

In some embodiments, the quantity of the carbon-carbon bond, the carbon-oxygen bond, the carbon-nitrogen bond, the nitrogen-oxygen bond, the phosphorus-oxygen bond, or the combination thereof is 52.

In some embodiments, the quantity of the carbon-carbon bond, the carbon-oxygen bond, the carbon-nitrogen bond, the nitrogen-oxygen bond, the phosphorus-oxygen bond, or the combination thereof is 53.

In some embodiments, the quantity of the carbon-carbon bond, the carbon-oxygen bond, the carbon-nitrogen bond, the nitrogen-oxygen bond, the phosphorus-oxygen bond, or the combination thereof is 54.

In some embodiments, the carbon-carbon bond, the carbon-oxygen bond, the carbon-nitrogen bond, the nitrogen-oxygen bond, the phosphorus-oxygen bond, or the combination thereof exists in a glycol molecule, nitroindole, deoxyinosine, furan, or a phosphine oxide thereof.

In some embodiments, the glycol molecule is selected from ethylene glycol, propylene glycol, butylene glycol, pentylene glycol, hexylene glycol, nonamethylene glycol, dodecamethylene glycol, or polyethylene glycol.

In some embodiments, the polyethylene glycol is selected from triethylene glycol, tetraethylene glycol, hexaethylene glycol, polyethylene glycol 2000, or polyethylene glycol 4500.

In some embodiments, the blocker further contains a phosphodiester bond.

In some embodiments, the blocker includes the following structural formula, m being an integer from 3-150:

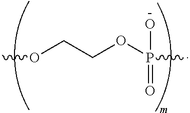

In some embodiments, m is 6.

In some embodiments, m is 7.

In some embodiments, m is 8.

In some embodiments, m is 9.

In some embodiments, m is 10.

In some embodiments, the blocker includes the following structural formula, n being an integer from 4-150:

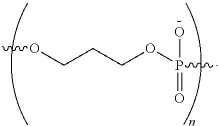

In some embodiments, n is 5.

In some embodiments, n is 6.

In some embodiments, n is 7.

In some embodiments, n is 8.

In some embodiments, n is 9.

In some embodiments, the blocker includes the following structural formula, a being an integer from 3-150:

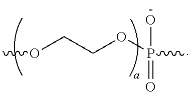

In some embodiments, a is 3.

In some embodiments, a is 4.

In some embodiments, a is 5.

In some embodiments, a is 6.

In some embodiments, a is 7.

In some embodiments, a is 8.

In some embodiments, a is 9.

In some embodiments, a is 10.

In some embodiments, a is 45.

In some embodiments, a is 46.

In some embodiments, a is 102.

In some embodiments, a is 103.

In some embodiments, the basic sequence is selected from SEQ ID NO: 1, SEQ ID NO: 2, SEQ ID NO: 3, or SEQ ID NO: 4.

In some embodiments, the basic sequence is selected from SEQ ID NO: 1.

In some embodiments, the basic sequence is selected from SEQ ID NO: 2.

In some embodiments, the basic sequence is selected from SEQ ID NO: 3.

In some embodiments, the basic sequence is selected from SEQ ID NO: 4.

In some embodiments, the basic sequence further includes a modified base, and the modified base is selected from a bicyclic nucleotide modified base, a phosphorothioate modified base, a 2′-methoxy modified base, or a combination thereof.

In some embodiments, the quantity of the modified base is 1-20.

In some embodiments, the quantity of the modified base is 2-18.

In some embodiments, the quantity of the modified base is 3-16.

In some embodiments, the bicyclic nucleotide modified base is selected from a locked nucleic acid modified base.

In some embodiments, the locked nucleic acid modified base is selected from LNA-A, LNA-G, LNA-C, LNA-T, or a combination thereof.

In some embodiments, the locked nucleic acid modified base is LNA-A, LNA-C, or LNA-T.

In some embodiments, the phosphorothioate modified base is selected from phosphorothioate modified adenine, phosphorothioate modified guanine, phosphorothioate modified cytosine, phosphorothioate modified thymine, phosphorothioate modified uracil, or a combination thereof.

In some embodiments, preferably, the phosphorothioate modified base is selected from phosphorothioate modified adenine, phosphorothioate modified cytosine, phosphorothioate modified thymine, phosphorothioate modified uracil, or a combination thereof.

In some embodiments, the 2′-methoxy modified base is selected from 2′-methoxy modified adenine, 2′-methoxy modified guanine, 2′-methoxy modified cytosine, 2′-methoxy modified thymine, 2′-methoxy modified uracil, or a combination thereof.

In some embodiments, preferably, the 2′-methoxy modified base is selected from 2′-methoxy modified guanine, 2′-methoxy modified cytosine, 2′-methoxy modified uracil, or a combination thereof.

In some embodiments, the oligonucleotide further includes a 3′-end modification. In some embodiments, in the oligonucleotide, the 3′-end modification is selected from 3′-Spacer C3 modification or 3′-ddC modification.

According to another aspect, the present application provides a combination of oligonucleotide sequences, including at least two oligonucleotides.

In some embodiments, the combination of oligonucleotide sequences includes two oligonucleotides.

In some embodiments, the combination of oligonucleotide sequences includes an oligonucleotide as set forth in SEQ ID NO: 5 and an oligonucleotide as set forth in SEQ ID NO: 6.

In some embodiments, the combination of oligonucleotide sequences includes an oligonucleotide as set forth in SEQ ID NO: 7 and an oligonucleotide as set forth in SEQ ID NO: 8.

In some embodiments, the combination of oligonucleotide sequences includes an oligonucleotide as set forth in SEQ ID NO: 9 and an oligonucleotide as set forth in SEQ ID NO: 10.

In some embodiments, the combination of oligonucleotide sequences includes an oligonucleotide as set forth in SEQ ID NO: 11 and an oligonucleotide as set forth in SEQ ID NO: 12.

In some embodiments, the combination of oligonucleotide sequences includes an oligonucleotide as set forth in SEQ ID NO: 13 and an oligonucleotide as set forth in SEQ ID NO: 14.

In some embodiments, the combination of oligonucleotide sequences includes an oligonucleotide as set forth in SEQ ID NO: 15 and an oligonucleotide as set forth in SEQ ID NO: 16.

According to still another aspect, the present application provides a kit, including the oligonucleotide or the combination of oligonucleotide sequences.

Further, the present application provides use of the oligonucleotide, the combination of oligonucleotide sequences, or the kit in sequencing.

In some specific embodiments, the use in sequencing is high-throughput sequencing, polynucleotide sequencing, forensic science, disease detection, medical diagnosis, precision medicine, companion diagnostics, non-invasive prenatal testing, or early tumor screening.

### Term Explanation:

Oligonucleotide: the oligonucleotide of the present application refers to a polynucleotide chain with a relatively small length, preferably a single strand. The length of the oligonucleotide is usually less than 200 residues (for example, between 15 and 100), and preferably the length of the basic sequence in the oligonucleotide is 58-64 residues. The oligonucleotide further includes a blocker. The backbone of the blocker contains a carbon-carbon bond, a carbon-oxygen bond, a carbon-nitrogen bond, a nitrogen-oxygen bond, or a combination thereof, and the quantity of the carbon-carbon bond, the carbon-oxygen bond, the carbon-nitrogen bond, the nitrogen-oxygen bond, or the combination thereof is 4-150.

Backbone: a natural or non-natural linear modification structure inserted between two natural nucleotides in an oligonucleotide sequence containing a blocker. For example, the backbone of an ethylene glycol phosphodiester bond structure contains 2 carbon-oxygen bonds, 1 carbon-carbon bond, and 2 phosphorus-oxygen bonds; the backbone of a propylene glycol phosphodiester bond structure contains 2 carbon-oxygen bonds, 2 carbon-carbon bonds, and 2 phosphorus-oxygen bonds; and the backbone of a hexaethylene glycol phosphodiester bond structure contains 12 carbon-oxygen bonds, 6 carbon-carbon bonds, and 2 phosphorus-oxygen bonds.

The ethylene glycol phosphodiester bond structure: a structural unit of a chain finally formed after solid-phase synthesis of a raw material of ethylene glycol phosphoramidite monomer, with a structural formula of:

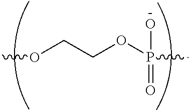

The propylene glycol phosphodiester bond structure: a structural unit of a chain finally formed after solid-phase synthesis of a raw material of propylene glycol phosphoramidite monomer, with a structural formula of:

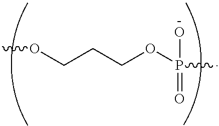

The hexaethylene glycol phosphodiester bond structure: a structural unit of a chain finally formed after solid-phase synthesis of a raw material of hexaethylene glycol phosphoramidite monomer, with a structural formula of:

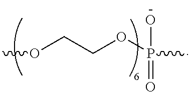

Bicyclic nucleotide modified base: a nucleotide-like derivative, the oxygen atom at position 2′ and the carbon atom at position 4′ in the ribonucleotide are covalently linked through a chemical bond.

Locked nucleic acid modified base: one of bicyclic nucleotide modified bases, it is a base derivative with a rigid structure formed by dehydration at positions 2′-O and 4′-C of β-D-ribofuranose. For example, the locked nucleic acid modified base includes locked nucleic acid modified adenine (LNA-A, LA for short), locked nucleic acid modified cytosine (LNA-C, LC for short), locked nucleic acid modified guanine (LNA-G, LG for short), and locked nucleic acid modified thymine (LNA-T, LT for short).

Phosphorothioate modification: the phosphorothioate modification is carried out by substituting one oxygen atom in the phosphodiester bond between single nucleotides with sulfur. Binding modification is carried out after the base is added. The common double bond “O” in the phosphate between two bases can be substituted with the double bond “S”.

2′-methoxy modification: a nucleotide derivative in which the hydrogen atom connected to the carbon atom at position 2′ of pentose in the deoxynucleotide is substituted with the methoxy.

3′-Spacer C3 structure: 3′-Spacer C3 is used to introduce a 3′ spacer arm to prevent 3′-end exonuclease and 3′-end polymerase from functioning.

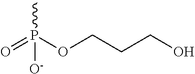

3′-ddC structure: 3′ddC Dideoxy-C is linked at the end to prevent the DNA polymerase from extending at the end.

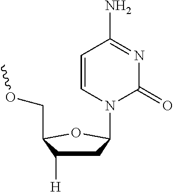

The “sample” in the present application is used according to its broadest meaning. In one meaning, it includes a samples or cultures obtained from any source, preferably a biological source (including eukaryotes or prokaryotes). Biological samples may be obtained from animals (including humans) and include fluids, solids, and tissues. Biological samples include blood products, such as plasma and serum, from non-human primates, sheep, cattle, ruminants, rabbits, pigs, goats, horses, dogs, cats, poultry, etc. In addition, the samples used herein include biological samples from plants, for example, samples from any organism in the plantae (such as monocotyledon or dicotyledon). The samples may also be from fungi, algae, bacteria, etc. It is expected that the present application does not limit the source of the samples. The samples used herein are usually “samples of nucleic acid”, “nucleic acid samples”, “target nucleic acid samples”, or “target samples” containing nucleic acid from any source (such as DNA, RNA, cDNA, mRNA, tRNA, miRNA, and rRNA).

Capture efficiency: the number of bases in the target region included in the sequencing reads/the number of all bases in the sequencing reads.

“Library” or “target sequence” refers to specific target nucleic acid (deoxyribonucleic acid or ribonucleic acid) sequences used in research, isolation, amplification, or other processes, including single-stranded sequences, double-stranded sequences, or their complementary sequences, and also including one type of in vitro synthesis products converted from another type of nucleic acid molecules (such as DNA, RNA, and cDNA) and synthetic molecules containing nucleotide analogs.

### Beneficial Effect

The present application provides oligonucleotide containing a blocker. The use of the oligonucleotide or a combination thereof can not only have a good blocking effect on the adapter sequence when the target sequence in a single sample is captured, reducing non-specific capture and improving capture efficiency, but also particularly effectively block the adapter sequences at both ends of the target sequences in a plurality of samples, improving the capture efficiency of the target sequence. In addition, the universal blocking oligonucleotide provided in the present application includes at least two short nucleic acid sequences, which can significantly reduce the production cost and cycle compared with similar products on the market.

### BRIEF DESCRIPTION OF THE DRAWINGS

FIG. 1 is a diagram of blocking of an oligonucleotide containing a blocker.

FIG. 2 is an analysis diagram of a relative content of SEQ ID NO: 5 measured by HPLC in Example 1.

FIG. 3 is an analysis diagram of actual relative molecular mass measurement of SEQ ID NO: 5 prepared in Example 1.

FIG. 4 is an analysis diagram of a relative content of SEQ ID NO: 6 measured by HPLC in Example 1.

FIG. 5 is an analysis diagram of actual relative molecular mass measurement of SEQ ID NO: 6 prepared in Example 1.

FIG. 6 is an analysis diagram of a relative content of SEQ ID NO: 7 measured by HPLC in Example 2.

FIG. 7 is an analysis diagram of actual relative molecular mass measurement of SEQ ID NO: 7 prepared in Example 2.

FIG. 8 is an analysis diagram of a relative content of SEQ ID NO: 8 measured by HPLC in Example 2.

FIG. 9 is an analysis diagram of actual relative molecular mass measurement of SEQ ID NO: 8 prepared in Example 2.

FIG. 10 is an analysis diagram of a relative content of SEQ ID NO: 9 measured by HPLC in Example 3.

FIG. 11 is an analysis diagram of actual relative molecular mass measurement of SEQ ID NO: 9 prepared in Example 3.

FIG. 12 is an analysis diagram of a relative content of SEQ ID NO: 10 measured by HPLC in Example 3.

FIG. 13 is an analysis diagram of actual relative molecular mass measurement of SEQ ID NO: 10 prepared in Example 3.

FIG. 14 is an analysis diagram of a relative content of SEQ ID NO: 11 measured by HPLC in Example 4.

FIG. 15 is an analysis diagram of actual relative molecular mass measurement of SEQ ID NO: 11 prepared in Example 4.

FIG. 16 is an analysis diagram of a relative content of SEQ ID NO: 12 measured by HPLC in Example 4.

FIG. 17 is an analysis diagram of actual relative molecular mass measurement of SEQ ID NO: 12 prepared in Example 4.

FIG. 18 is an analysis diagram of a relative content of SEQ ID NO: 13 measured by HPLC in Example 5.

FIG. 19 is an analysis diagram of actual relative molecular mass measurement of SEQ ID NO: 13 prepared in Example 5.

FIG. 20 is an analysis diagram of a relative content of SEQ ID NO: 14 measured by HPLC in Example 5.

FIG. 21 is an analysis diagram of actual relative molecular mass measurement of SEQ ID NO: 14 prepared in Example 5.

FIG. 22 is an analysis diagram of a relative content of SEQ ID NO: 15 measured by HPLC in Example 6.

FIG. 23 is an analysis diagram of actual relative molecular mass measurement of SEQ ID NO: 15 prepared in Example 6.

FIG. 24 is an analysis diagram of a relative content of SEQ ID NO: 16 measured by HPLC in Example 6.

FIG. 25 is an analysis diagram of actual relative molecular mass measurement of SEQ ID NO: 16 prepared in Example 6.

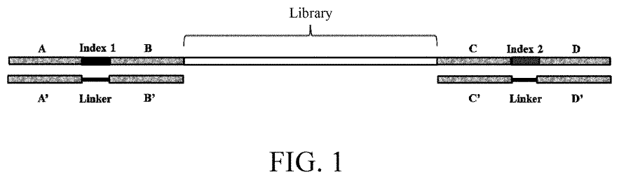

### SEQUENCE LISTING

The Sequence Listing is submitted as an ASCII text file Sequence_Listing.txt, generated on Jun. 3, 2022, 22,663 bytes, which is herein incorporated by reference in its entirety.

SEQ ID NOS: 1-4 are exemplary basic oligonucleotide sequences.

SEQ ID NOS: 5-16 are an exemplary oligonucleotide sequences containing a blocker.

### DETAILED DESCRIPTION

Preparation of reagents, raw materials, and apparatus:

A deprotection reagent was prepared by dissolving dichloroacetic acid in a dichloromethane solution (3%, v/v).

An activator was prepared by dissolving 5-ethylthio-1H-tetrazole in an acetonitrile solution (0.25 M).

A capping reagent A was prepared by dissolving N-methylimidazole in an acetonitrile solution (20%, v/v).

A capping reagent B was prepared by dissolving acetic anhydride in an acetonitrile solution (30%, v/v).

An oxidizing agent (0.06 M) was prepared by dissolving iodine in a pyridine solution/(a mixed solution of pyridine and acetonitrile (3/2, v/v) of 3-((dimethylamino-methylene)amino)-3H-1,2,4-dithiazole-3-thione (0.06 M)).

Phosphoramidite monomer (A/T/G/C/mA/mG/mC/mU) solutions (0.067 M) were separately prepared by separately dissolving phosphoramidite monomers (A/T/G/C/mA/mG/mC/mU) in acetonitrile solutions.

Locked nucleic acid phosphoramidite monomer (LNA-A/LNA-G/LNA-C/LNA-T) solutions were separately prepared by dissolving LNA-C (0.067 M) in a mixed solution of acetonitrile and dichloromethane (1/1, v/v) and separately dissolving LNA-A/LNA-G/LNA-T (0.067 M) in acetonitrile solutions.

Raw materials (0.067 M) for synthesizing a blocker were prepared by separately dissolving an ethylene glycol phosphoramidite monomer, a propylene glycol phosphoramidite monomer, and a hexaethylene glycol phosphoramidite monomer in acetonitrile solutions.

Solid-phase synthesis supports: 3′-Spacer C3-CPG support: 200 nmol/piece; and 3′-ddC-CPG support: 200 nmol/piece.

Apparatus: Dr. Oligo 96/192 synthesizer.

### Example 1 Synthesis of Oligonucleotide Sequence Containing Blocker

SEQ ID NO: 5
5′-AATGATACGGCGACCACCGAGATCTACACNACACTCTTTCCCTACAC
GACGCTCTTCCGATCT-3′

The LC modification positions (starting from the 5′-end) are 8, 11, 14, 17, 24, 32, 34, 36, 40, 42, 45, 47, 50, 52, 54, and 58.

Nis a hexaethylene glycol phosphodiester bond structure.

SEQ ID NO: 6
5′-AGATCGGAAGAGCACACGTCTGAACTCCAGTCACNATCTCGTATGCC
GTCTTCTGCTTG-3′

The LC modification positions (starting from the 5′-end) are 5, 13, 15, 17, 20, 25, 27, 28, 32, 38, 40, 46, 47, 50, 53, and 56.

N is a hexaethylene glycol phosphodiester bond structure.

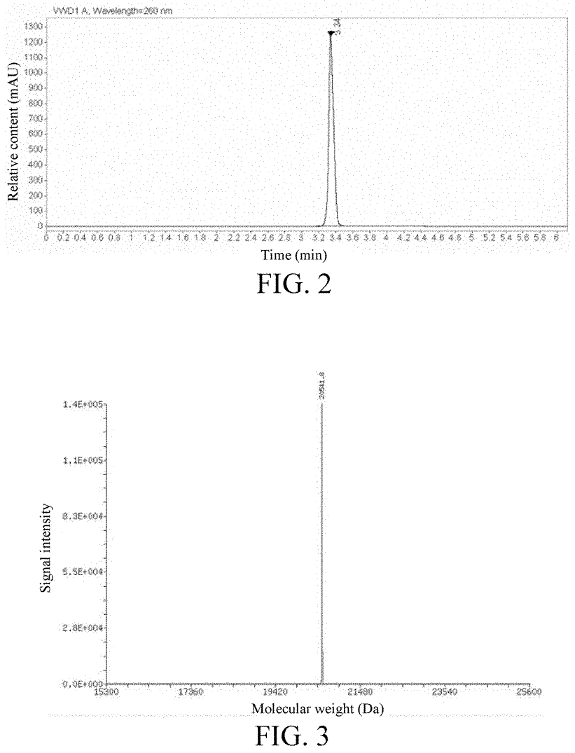

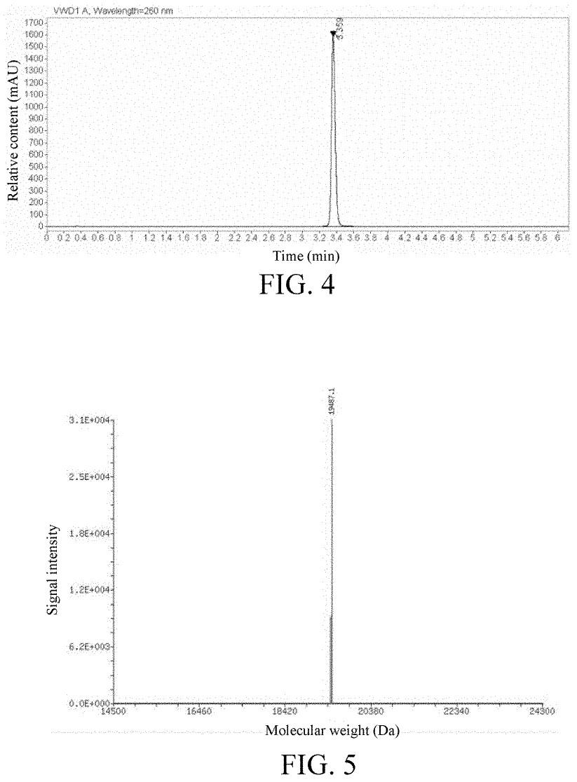

Solid-phase synthesis steps:
1. The foregoing reagents were prepared on the Dr. Oligo192 apparatus, and the air tightness, channel unobstructedness, and operating state of the apparatus were checked. After there was no error in the check, a 3′-Spacer C3-CPG support with a pore size of 1000 Å, a sample load of 26 μmol/g, and a synthesis scale of 200 nmol was selected to synthesize the foregoing two primers in a synthesis direction from the 3′-end to the 5′-end with reference to the sequence synthesis procedures and methods in Current Protocols in Nucleic Acid Chemistry (2001) 3.8.1-3.8.15.
2. After the synthesis, the support was transferred into a 2 mL screw-cap centrifuge tube with a corresponding label, and 1 mL of ice-cold NH₃·H₂O was added, to react in an oven at 80° C. for 2 h.
3. After the reaction, the centrifuge tube was taken out to cool down to room temperature, the cap was slowly unscrewed, centrifugation was first carried out and vacuumizing was then slowly started at 60° C. by using a fast centrifugal concentration dryer, the pressure was reduced to quickly volatilize the ammonia water and remove the ammonia smell, the CPG support was removed through filtration with a 0.22 μm membrane, and the filtrate was transferred into a new 2 mL screw-cap centrifuge tube with a corresponding label and concentrated under reduced pressure to a dry powder.
4. The dry powder in step 3 was added into 1 mL of Milli-Q water to shake for 15s and to be centrifuged at a high speed of 12000 rpm for 15s and mixed well for use. A 96-Well ELISA plate was used. 195 μL of deionized water was used as a blank control to measure the blank absorbance after calibration. 5 μL of the mixed sample solution was drawn up and dispensed slowly to mix well 5 times by using a pipette, and then the absorbance after calibration was measured by using the Tecan ELISA reader (Infinite® 200 PRO). The difference between the two was the sample absorbance. The crude product amount (unit: nmol) was calculated according to the corresponding molar attenuation coefficient, dilution factor, and remaining volume of the sample.
5. The crude product containing two blocking oligonucleotides was filtered by a 0.22 μm membrane. Target fractions were respectively separated and collected by using the Waters 1525 high-performance liquid preparative chromatography (model: XBridge BEH C18 OBD preparative column; pore size: 130 A; particle size: 5μm; length: 10 mm×100 mm; mobile phase A: Milli-Q aqueous solution containing 100 mM of triethylammonium acetate (pH 8.5); mobile phase B: mixed solution of the mobile phase A and acetonitrile (5/95, v/v); gradient: 6%-16%; time: 0-12 min; flow rate: 3 mL/min; temperature: room temperature). The target purity was analyzed from 0.1 nmol of the fraction by the Agilent 1260 high-performance liquid chromatography (model: XBridge Oligonucleotide BEH C18 Column; pore size: 130 A; particle size: 2.5 μm; length: 4.6 mm×50 mm; mobile phase A: Milli-Q aqueous solution containing 100 mM of triethylammonium acetate (pH 7.0); mobile phase B: mixed solution of the mobile phase A and acetonitrile (5/95, v/v); gradient: 5%-25%; time: 0-6 min; flow rate: 2 mL/min; temperature: room temperature), and the target mass was analyzed by MS (Thermo, LTQ). The remaining fractions were concentrated under reduced pressure to a dry powder and then redissolved with Milli-Q water. The samples that meet the QC standard (the HPLC purity is greater than 90%, the MS target peak ratio is greater than 90%, and the single impurity content is less than 5%) were quantified by the Tecan ELISA reader. The qualified samples were diluted to 1 nmol/μL and then stored at −20° C. For target sequence hybridization capture, the oligonucleotide 1 and oligonucleotide 2 were mixed in equal volumes and centrifuged to mix well to form a universal blocking oligonucleotide mixture 1. The purities of the two synthetic oligonucleotides are shown in FIG. 2 and FIG. 4. The theoretical molecular weight (MW) of SEQ ID NO: 5 is 20542. As shown in FIG. 3, the MW of actually prepared SEQ ID NO: 5 is 20541.8. The theoretical MW of SEQ ID NO: 6 is 19488. As shown in FIG. 5, the MW of actually prepared SEQ ID NO: 6 is 19487.1.

### Example 2 Synthesis of Oligonucleotide Sequence Containing Blocker

SEQ ID NO: 7
5′-AATGATACGGCGACCACCGAGATCTACACNNNNNNNNACACTCTTTC
CCTACACGACGCTCTTCCGATCT-3′

The LC modification positions (starting from the 5-end) are 8, 11, 14, 17, 24, 39, 41, 43, 47, 49, 52, 54, 57, 59, 61, and 65.

N is an ethylene glycol phosphodiester bond structure.

SEQ ID NO: 8
5′-AGATCGGAAGAGCACACGTCTGAACTCCAGTCACNNNNNNNNATCT
CGTATGCCGTCTTCTGCTTG-3′

The LC modification positions (starting from the 5-end) are 5, 13, 15, 17, 20, 25, 27, 28, 32, 45, 47, 53, 54, 57, 60, and 63.

Nis an ethylene glycol phosphodiester bond structure.

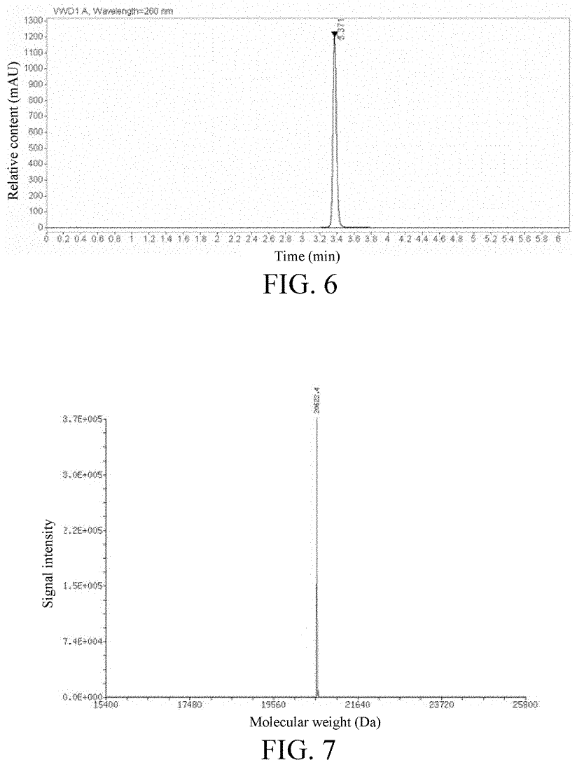

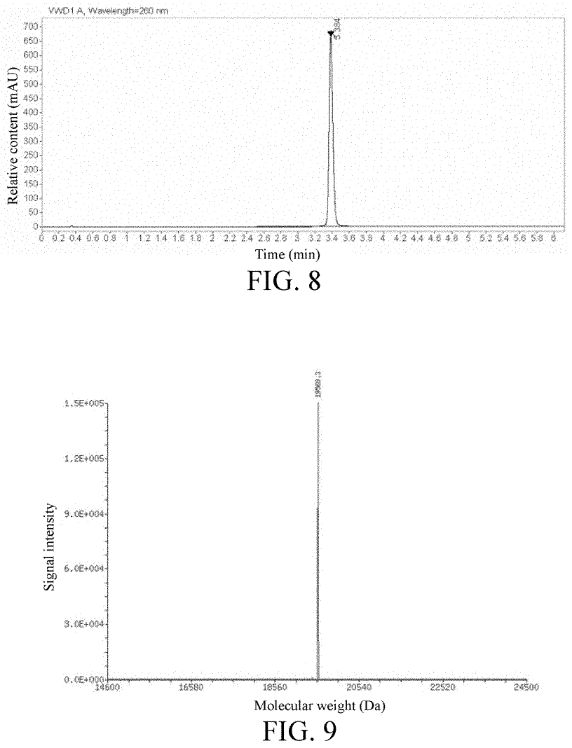

Referring to the method in Example 1, a blocking oligonucleotide combination 2 was obtained by solid-phase synthesis and purification. The two blocking oligonucleotides therein were mixed in equal volumes and centrifuged to mix well to form a universal blocking oligonucleotide mixture 2. The purities of the two synthetic oligonucleotides are shown in FIG. 6 and FIG. 8. The theoretical molecular weight (MW) of SEQ ID NO: 7 is 20624. As shown in FIG. 7, the MW of actually prepared SEQ ID NO: 7 is 20622.4. The theoretical MW of SEQ ID NO: 8 is 19568. As shown in FIG. 9, the MW of actually prepared SEQ ID NO: 8 is 19569.3.

### Example 3 Synthesis of Oligonucleotide Sequence Containing Blocker

SEQ ID NO: 9
5′-AATGATACGGCGACCACCGAGATCTACACNNNNNNNACACTCTTTCC
CTACACGACGCTCTTCCGATCT-3′

The LC modification positions (starting from the 5-end) are 8, 11, 14, 17, 24, 38, 40, 42, 46, 48, 51, 53, 56, 58, 60, and 64.

N is a propylene glycol phosphodiester bond structure.

SEQ ID NO: 10
5′-AGATCGGAAGAGCACACGTCTGAACTCCAGTCACNNNNNNNATCTCG
TATGCCGTCTTCTGCTTG-3′

The LC modification positions (starting from the 5-end) are 5, 13, 15, 17, 20, 25, 27, 28, 32, 44, 46, 52, 53, 56, 59, and 62.

N is a propylene glycol phosphodiester bond structure.

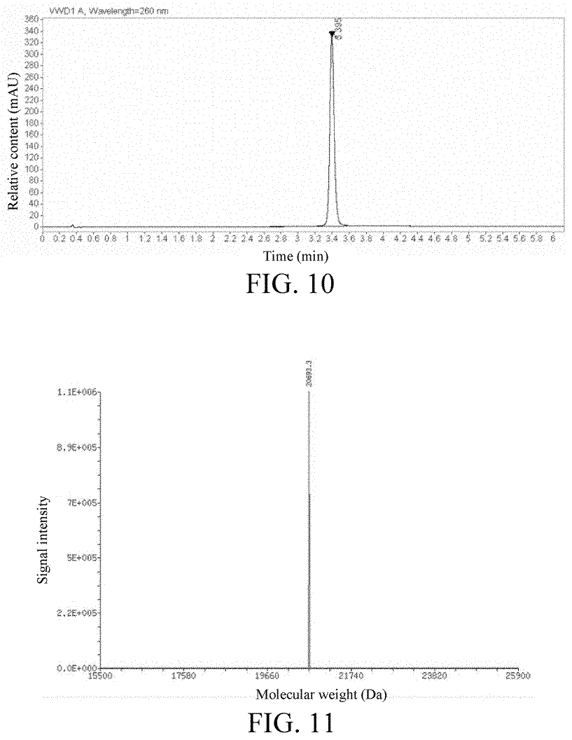

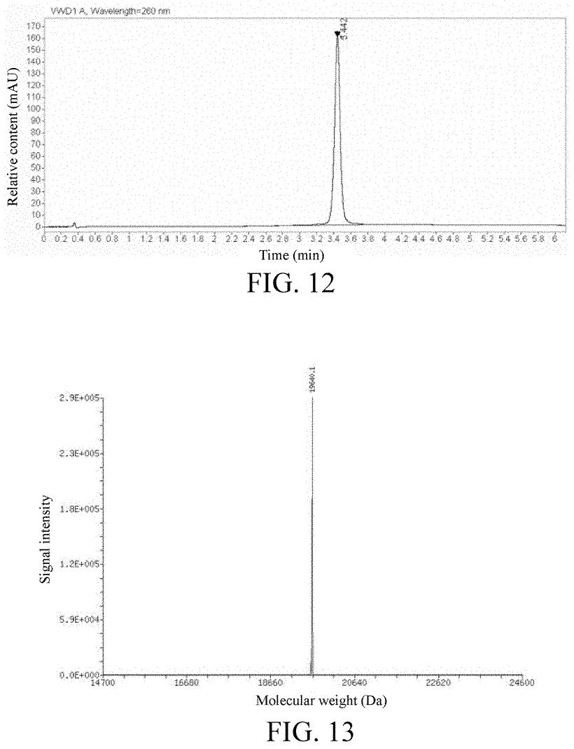

Referring to the method in Example 1, a blocking oligonucleotide combination 3 was obtained by solid-phase synthesis and purification. The two blocking oligonucleotides therein were mixed in equal volumes and centrifuged to mix well to form a universal blocking oligonucleotide mixture 3. The purities of the two synthetic oligonucleotides are shown in FIG. 10 and FIG. 12. The theoretical molecular weight (MW) of SEQ ID NO: 9 is 20694. As shown in FIG. 11, the MW of actually prepared SEQ ID NO: 9 is 20693.3. The theoretical MW of SEQ ID NO: 10 is 19639. As shown in FIG. 13, the MW of actually prepared SEQ ID NO: 10 is 19640.1.

### Example 4 Synthesis of Oligonucleotide Sequence Containing Blocker

SEQ ID NO: 11
5′-
CTGTCTCTTATACACATCTCCGAGCCCACGAGACNNNNNNNNATCTCGT
ATGCCGTCTTCTGCTTG-3′

The LC modification positions (starting from the 5-end) are 5, 7, 13, 15, 18, 21, 25, 27, 29, 34, 45, 47, 54, 57, 60, and 63.

N is an ethylene glycol phosphodiester bond structure.

SEQ ID NO: 12
5′-CTGTCTCTTATACACATCTGACGCTGCCGACGANNNNNNNNGTGTAG
ATCTCGGTGGTCGCCGTATCATT-3′

The LC modification positions (starting from the 5-end) are 5, 7, 13, 15, 18, 22, 24, 28, 31, 50, 52, 59, 61, and 67. The LT modification positions (starting from the 5-end) are 45 and 55.

N is an ethylene glycol phosphodiester bond structure.

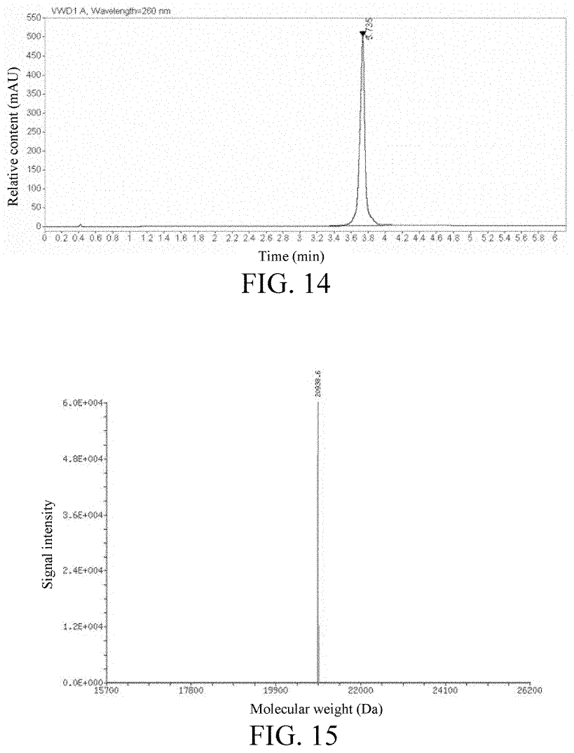

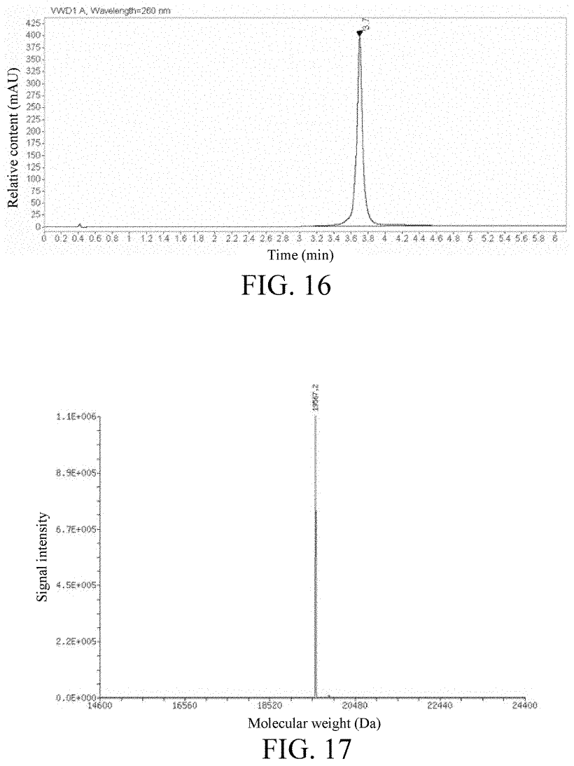

Referring to the method in Example 1, a blocking oligonucleotide combination 4 was obtained by solid-phase synthesis and purification. The two blocking oligonucleotides therein were mixed in equal volumes and centrifuged to mix well to form a universal blocking oligonucleotide mixture 4. The purities of the two synthetic oligonucleotides are shown in FIG. 14 and FIG. 16. The theoretical molecular weight (MW) of SEQ ID NO: 11 is 20942. As shown in FIG. 16, the MW of actually prepared SEQ ID NO: 11 is 20938.6. The theoretical MW of SEQ ID NO: 12 is 19570. As shown in FIG. 5, the MW of actually prepared SEQ ID NO: 12 is 19567.2.

### Example 5 Synthesis of Oligonucleotide Sequence Containing Blocker

SEQ ID NO: 13
5′-mC*mU*GTCTCTTATACACATCTCCGAGCCCACGAGACNNNNNNNNA
TCTCGTATGCCGTCTTCTGCT*mU*mG-3′

The LC modification positions (starting from the 5-end) are 5, 7, 13, 15, 18, 21, 25, 27, 29, 34, 45, 47, 54, 57, 60, and 63.

N is an ethylene glycol phosphodiester bond structure, and * is phosphorothioate modification.

SEQ ID NO: 14
5′-mC*mU*GTCTCTTATACACATCTGACGCTGCCGACGANNNNNNNNGT
GTAGATCTCGGTGGTCGCCGTATCA*mU*mU-3′

The LC modification positions (starting from the 5-end) are 5, 7, 13, 15, 18, 22, 24, 28, 31, 50, 52, 59, 61, and 67. The LT modification positions (starting from the 5-end) are 45 and 55.

N is an ethylene glycol phosphodiester bond structure, and * is phosphorothioate modification.

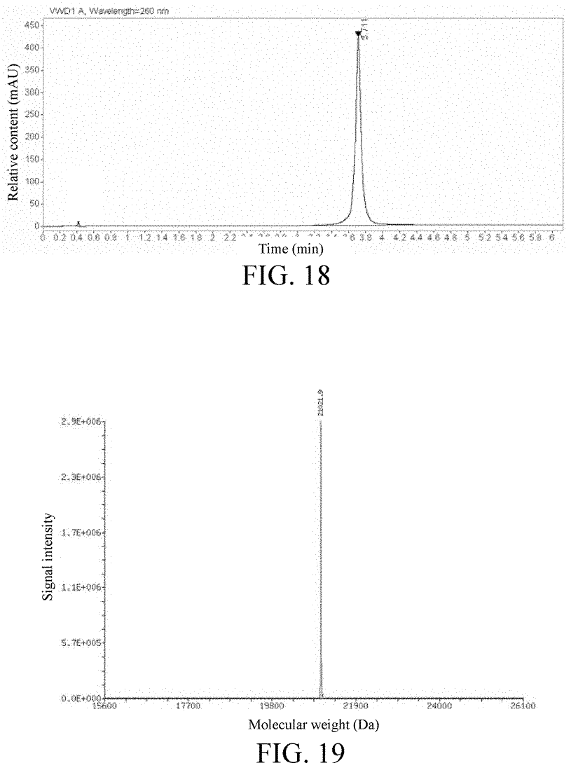

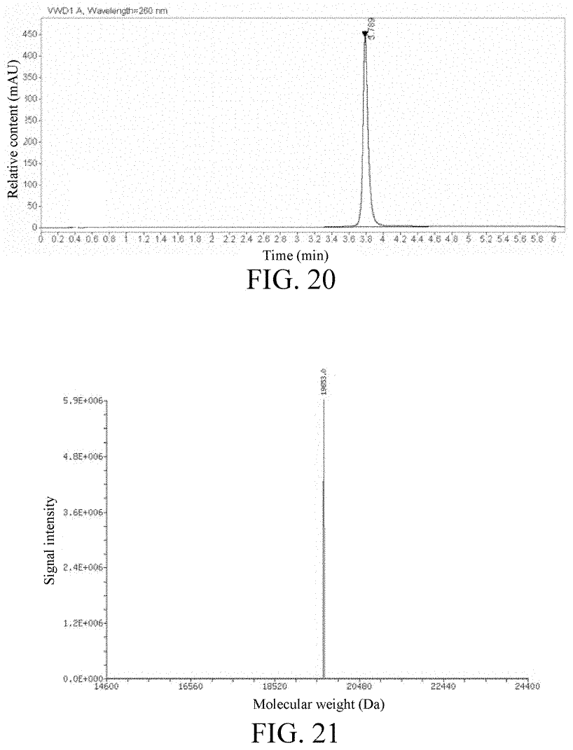

Referring to the method in Example 1, a blocking oligonucleotide combination 5 was obtained by solid-phase synthesis and purification. The two blocking oligonucleotides therein were mixed in equal volumes and centrifuged to mix well to form a universal blocking oligonucleotide mixture 5. The purities of the two synthetic oligonucleotides are shown in FIG. 18 and FIG. 20. The theoretical molecular weight (MW) of SEQ ID NO: 13 is 21024. As shown in FIG. 19, the MW of actually prepared SEQ ID NO: 13 is 21021.9. The theoretical MW of SEQ ID NO: 14 is 19650. As shown in FIG. 21, the MW of actually prepared SEQ ID NO: 14 is 19653.0.

### Example 6 Synthesis of Oligonucleotide Sequence Containing Blocker

SEQ ID NO: 15
5′-CTGTCTCTTATACACATCTCCGAGCCCACGAGACNNNNNNNATCTCG
TATGCCGTCTTCTGCTTG-3′

The LC modification positions (starting from the 5-end) are 5, 7, 13, 15, 18, 21, 25, 27, 29, 34, 44, 46, 53, 56, 59, and 62.

N is a propylene glycol phosphodiester bond structure.

SEQ ID NO: 16
5′-CTGTCTCTTATACACATCTGACGCTGCCGACGANNNNNNNGTGTAGA
TCTCGGTGGTCGCCGTATCATT-3′

The LC modification positions (starting from the 5-end) are 5, 7, 13, 15, 18, 22, 24, 28, 31, 49, 51, 58, 60, and 66. The LT modification positions (starting from the 5-end) are 44 and 54.

N is a propylene glycol phosphodiester bond structure.

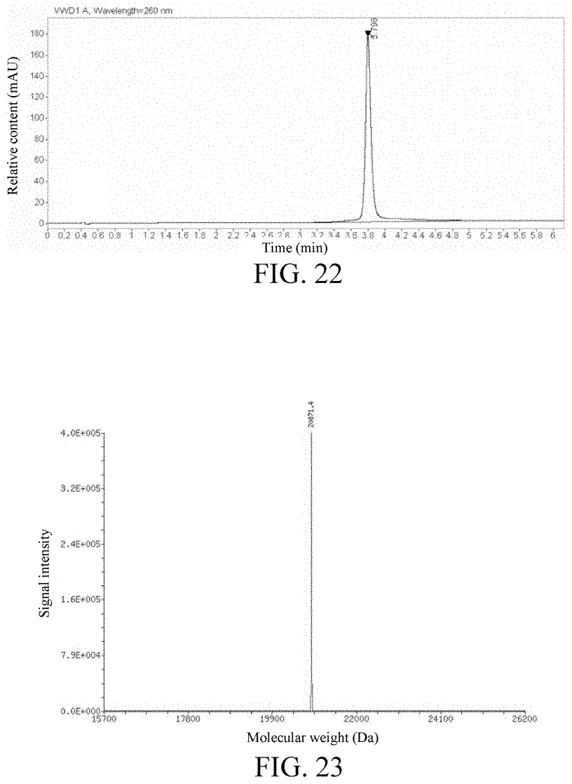

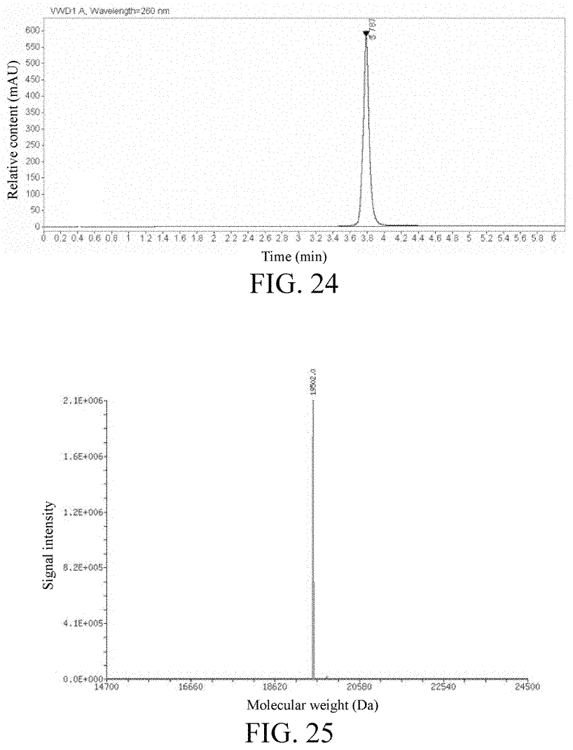

Referring to the method in Example 1, a blocking oligonucleotide combination 6 was obtained by solid-phase synthesis and purification. The two blocking oligonucleotides therein were mixed in equal volumes and centrifuged to mix well to form a universal blocking oligonucleotide mixture 6. The purities of the two synthetic oligonucleotides are shown in FIG. 22 and FIG. 24. The theoretical molecular weight (MW) of SEQ ID NO: 15 is 20870. As shown in FIG. 23, the MW of actually prepared SEQ ID NO: 15 is 20871.4. The theoretical MW of SEQ ID NO: 16 is 19504. As shown in FIG. 25, the MW of actually prepared SEQ ID NO: 16 is 19502.0.

### Example 7 Use of Combination of Oligonucleotide Sequence Containing Blocker in Target Sequence Hybridization Capture

1. Hybridization reaction

1.1. Preparation of the following blocking mixture:

| Blocking mixture | Volume (μL) |
|---|---|
| Human Cot DNA (1 μg/μL) | 5 |
| Universal blocking oligonucleotide mixture | 2 |

The corresponding “universal blocking oligonucleotide mixture” was not added to the blocking mixture in a negative control group.

1.2. 7 μL of the blocking mixture was transferred into a PCR tube to capture a library, where each 7 μL of the blocking mixture corresponds to a 500 ng library.

1.3. The mixture in the PCR tube was concentrated in a vacuum to no liquid.

1.4. The hybridization elution kit produced by IDT (Integrated DNA Technologies, Inc.) was taken out in advance and equilibrated at room temperature.

1.5. Preparation of hybridization mixture:

| Hybridization mixture | Volume (μL) |
|---|---|
| 2× hybridization buffer | 8.5 |
| Hybridization enhancer | 2.7 |
| Probe (3 pmol) | x |
| Nuclease-free water | 5.8 − x |
| Total volume | 17 |

1.6. After shaking, mixing, and centrifugation, 17 μL of the hybridization mixture was taken into the concentrated PCR tube.

1.7. After a short centrifugation and mixing, incubation was carried out at room temperature for at least 5 min.

1.8. The incubated PCR tube was placed on a PCR machine to run with the following hybridization procedures:

| Temperature | Time | |
|---|---|---|
| 95° C. | 30 s | Thermal |
| 65° C. | 16-18 h | cover: 100° C. |

2. Capture

2.1. 2×magnetic bead wash buffer, 10×wash buffer 1, 10×wash buffer 2, 10×wash buffer 3, 10×wash enhancement buffer, 2×hybridization buffer, and hybridization enhancer buffer were mixed well at room temperature and diluted to 1×working liquid for use (amount of each sample):

| | Nuclease-free | Buffer | Total volume |
|---|---|---|---|
| Composition | water (μL) | (μL) | (μL) |
| 2× magnetic bead wash buffer | 150 | 150 | 300 |
| 10× wash buffer 1 | 225 | 25 | 250 |
| 10× wash buffer 2 | 135 | 15 | 150 |
| 10× wash buffer 3 | 135 | 15 | 150 |
| 10× wash enhancement buffer | 270 | 30 | 300 |

2.2. Preparation of magnetic bead resuspension mixture:

| Magnetic bead resuspension | |
|---|---|
| mixture | Volume (μL) |
| 2× hybridization buffer | 8.5 |
| Hybridization enhancement | 2.7 |
| buffer | |
| Nuclease-free water | 5.8 |
| Total volume | 17 |

2.3. Magnetic beads were taken out, and shaking and mixing were carried out for 15s.

2.4. 50 μL of magnetic beads were taken into a 1.5 mL centrifuge tube, where each 50 μL of magnetic beads corresponds to a 500 ng library.

2.5. 100 μL of 1×magnetic bead wash buffer was added into the centrifuge tube to draw up and dispense 10 times for mixing.

2.6. The centrifuge tube was placed on a magnetic stand to stand for 1 min until the solution was clear, and then the supernatant was removed.

2.7. 100 μL of magnetic bead wash buffer was added into the centrifuge tube to draw up and dispense 10 times for mixing, the centrifuge tube was then placed on the magnetic stand to stand for 1 min until the solution was clear, and then the supernatant was removed.

2.8. The foregoing step was repeated for a total of 2 times of washing.

2.9. 17 μL of magnetic bead resuspension mixture was added into the centrifuge tube to draw up and dispense for mixing and centrifuged at 400 rpm for 10s, to ensure that there are no dried magnetic beads left on the tube wall.

2.10. After the hybridization reaction, 17 μL of the magnetic beads were transferred into the PCR tube to draw up and dispense for mixing, and in addition, the procedure of the PCR machine was set to 65° C. for constant (the thermal cover 70° C.).

2.11. The mixture was placed in the PCR machine to undergo the washing procedure at 65° C. for 45 min (need timing) and shaken quickly and slightly for mixing well every 10 min, and the wash buffer 1 and wash enhancement buffer were put into a water bath at 65° C. for at least 15 min.

2.12. After the 45 min timing was over, the sample was taken out from the PCR machine.

2.13. 100 μL of pre-heated wash buffer 1 was taken into the PCR tube to draw up and dispense 10 times for mixing to avoid bubbles. After the wash buffer 1 was used, the PCR tube was equilibrated at room temperature.

2.14. The sample was placed on the magnetic stand to stand for 1 min, and then the supernatant was removed.

2.15. 150 μL of pre-heated wash enhancement buffer was taken into the PCR tube to draw up and dispense 10 times for mixing to avoid bubbles.

2.16. The PCR tube was placed on the PCR machine to react for 5 min.

2.17. The sample was placed on the magnetic stand to stand for 1 min, and then the supernatant was removed.

2.18. 150 μL of pre-heated wash enhancement buffer was added to draw up and dispense

10 times for mixing and then react on the PCR machine for 5 min.

2.19. The sample was placed on the magnetic stand to stand for 1 min, and then the supernatant was removed.

3. Washing

3.1. 150 μL of wash buffer 1 at room temperature was added into a reaction tube to shake and mix thoroughly.

3.2. The incubation was carried out at room temperature for 2 min with shaking and mixing every 30s.

3.3. The sample after short centrifugation was placed on a magnetic stand to stand for 1 min until the solution was clear, and then the supernatant was removed.

3.4. 150 μL of wash buffer 2 at room temperature was added to shake and mix thoroughly.

3.5. The incubation was carried out at room temperature for 2 min with shaking and mixing every 30s.

3.6. The sample after short centrifugation was placed on a magnetic stand to stand for 1 min until the solution was clear, and then the supernatant was removed.

3.7. 150 μL of wash buffer 3 at room temperature was added to shake and mix thoroughly.

3.8. The incubation was carried out at room temperature for 2 min with shaking and mixing every 30s.

3.9. The sample after short centrifugation was placed on a magnetic stand to stand for 1 min until the solution was clear, and then the supernatant was removed.

3.10. The magnetic beads were dried (until there was no obvious residue of the wash buffer 3).

4. PCR after capture

4.1. Preparation of the following PCR mixture:

| PCR composition | Volume (μL) |
|---|---|
| 5× PCR buffer | 10 |
| 10 mM dNTP | 1 |
| Primer F (10 μM) | 1 |
| Primer R (10 μM) | 1 |
| DNA polymerase | 0.5 |
| Nuclease-free water | 36.5 |
| Total volume | 50 |

4.2. The PCR mixture was added into a PCR tube with dried magnetic beads to draw up and dispense for mixing.

4.3. Run with the following PCR procedures:

| | | Number of |
|---|---|---|
| Temperature | Time | cycles |
| 98° C. | 3 min | 1 |
| 98° C. | 20 s | 13 |
| 62° C. | 30 s | |
| 72° C. | 30 s | |
| 72° C. | 3 min | 1 |
| 4° C. | hold | 1 |

5. Magnetic bead purification

5.1. 1×(50 μL) Yeasen Beads were directly added into 50 μL of PCR reaction tube for magnetic bead purification, and finally the product was eluted with 21 μL of nuclease-free water.

One human genome NA12878 was used as a sample to prepare a library using the ABclonal Rapid DNA Lib Prep Kit, and then 500 ng of the library was used as a starting amount, the universal blocking oligonucleotide mixture obtained in Examples 1 to 6 was applied to obtain a hybridized capture library, respectively, with reference to the foregoing procedures of the hybridization reaction example. No blocking oligonucleotide was added in the negative control group during the hybridization capture. On-machine sequencing was performed by using the Nextseq next-generation sequencer, and the percentage of bases in the target region to total bases in the sequencing result was analyzed, to obtain the hybridization capture efficiency with the sample size of 1 as shown in Table 1. The result shows that, compared with the negative control group, the addition of the universal blocking oligonucleotide of the present application significantly improves the capture efficiency of the target sequence with the improvement rate of 144.8%-180.5%.

TABLE 1
Capture efficiency of target sequence with sample size of 1

| Group | Capture efficiency |
|---|---|
| Example 1 | 58.64% |
| Example 2 | 65.69% |
| Example 3 | 65.81% |
| Example 4 | 67.16% |
| Example 5 | 66.71% |
| Example 6 | 67.18% |
| Negative control | 23.95% |

8 human genomes NA12878 were used as samples. The ABclonal Rapid DNA Lib Prep Kit was used to separately prepare libraries. The 8 libraries were mixed at 62.5 ng each to ensure that the total amount of the library was 500 ng. The universal blocking oligonucleotide mixture obtained in Examples 2 and 3 was used to obtain a hybridized capture library with reference to the foregoing procedures of the hybridization reaction example. No blocking oligonucleotide was added in the negative control group during the hybridization capture. On-machine sequencing was performed by using the Nextseq next-generation sequencer, and the percentage of bases in the target region to total bases in the sequencing result was analyzed, to obtain the hybridization capture efficiency with the sample size of 8 as shown in Table 2. The result shows that, compared with the negative control group, the addition of the universal blocking oligonucleotide of the present application significantly improves the capture efficiency of the target sequence with the improvement rate of 160.5%-160.9%. In addition, comparing Table 1 and Table 2, it is found that the capture efficiency of the universal primer of the present application in multi-sample target sequence capture is not significantly different from the capture efficiency of the universal primer of the present application in single-sample target sequence capture. It can be learned from this that the universal blocking oligonucleotide provided by the present application has a significant specific capture effect in multi-sample target sequence collective capture, which can greatly reduce the operation time and cost of the target sequence library construction.

TABLE 2
Capture efficiency of target sequence with sample size of 8

| Group | Capture efficiency |
|---|---|
| Example 2 | 68.03% |
| Example 3 | 68.15% |
| Negative control | 26.12% |

1. An oligonucleotide containing a blocker introduced into a basic sequence, wherein the backbone of the blocker contains a carbon-carbon bond, a carbon-oxygen bond, a carbon-nitrogen bond, a nitrogen-oxygen bond, a phosphorus-oxygen bond, or a combination thereof, and the quantity of the carbon-carbon bond, the carbon-oxygen bond, the carbon-nitrogen bond, the nitrogen-oxygen bond, the phosphorus-oxygen bond, or the combination thereof is 10-900.

2-3. (Canceled)

4. The oligonucleotide according to claim 1, wherein the carbon-carbon bond, the carbon-oxygen bond, the carbon-nitrogen bond, the nitrogen-oxygen bond, the phosphorus-oxygen bond, or the combination thereof exists in a glycol molecule, nitroindole, deoxyinosine, furan, or a phosphine oxide thereof.

5. The oligonucleotide according to claim 4, wherein the glycol molecule is ethylene glycol, propylene glycol, butylene glycol, pentylene glycol, hexylene glycol, nonamethylene glycol, dodecamethylene glycol, or polyethylene glycol.

6. The oligonucleotide according to claim 1, wherein the blocker further contains a phosphodiester bond.

7. The oligonucleotide according to claim 6, wherein the blocker comprises the following structural formula, m being an integer from 3-150:

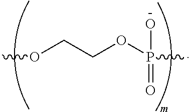

8. (Canceled)

9. The oligonucleotide according to claim 6, wherein the blocker comprises the following structural formula, n being an integer from 4-150:

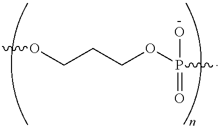

10. (Canceled)

11. The oligonucleotide according to claim 6, wherein the blocker comprises the following structural formula: 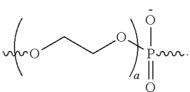
a being an integer from 3-150

12. The oligonucleotide according to any one of claims 1 to 11, wherein the basic sequence comprises is selected from SEQ ID NO: 1, SEQ ID NO: 2, SEQ ID NO: 3, or SEQ ID NO: 4.

13. The oligonucleotide according to claim 12, wherein the basic sequence further comprises a modified base, and the modified base is selected from a bicyclic nucleotide modified base, a phosphorothioate modified base, a 2′-methoxy modified base, or a combination thereof.

14. The oligonucleotide according to claim 13, wherein the quantity of the modified base is 1-20.

15-16. (Canceled)

17. The oligonucleotide according to claim 13, wherein the bicyclic nucleotide modified base is selected from a locked nucleic acid modified base; and wherein the locked nucleic acid modified base is LNA-A, LNA-G, LNA-C, LNA-T, or a combination thereof.

18-19. (Canceled)

20. The oligonucleotide according to claim 13, wherein the phosphorothioate modified base is phosphorothioate modified adenine, phosphorothioate modified guanine, phosphorothioate modified cytosine, phosphorothioate modified thymine, phosphorothioate modified uracil, or a combination thereof.

21. The oligonucleotide according to claim 13, wherein the 2′-methoxy modified base is 2′ methoxy modified adenine, 2′-methoxy modified guanine, 2′-methoxy modified cytosine, 2′-methoxy modified thymine, 2′-methoxy modified uracil, or a combination thereof.

22. The oligonucleotide according to claim 1, further comprising a 3′-end modification.

23. The oligonucleotide according to claim 22, wherein the 3′-end modification is a 3′Spacer C3 modification or 3′-ddC modification.

24. A combination of oligonucleotide sequences, comprising at least two oligonucleotides according to claim 1.

25. (Canceled)

26. The combination of oligonucleotide sequences according to claim 24, comprising:
an oligonucleotide as set forth in SEQ ID NO: 5 and an oligonucleotide as set forth in SEQ ID NO 6;
an oligonucleotide as set forth in SEQ ID NO: 7 and an oligonucleotide as set forth in SEQ ID NO: 8;
an oligonucleotide as set forth in SEQ ID NO: 9 and an oligonucleotide as set forth in SEQ ID NO: 10;
an oligonucleotide as set forth in SEQ ID NO: 11 and an oligonucleotide as set forth in SEQ ID NO: 12;
an oligonucleotide as set forth in SEQ ID NO: 13 and an oligonucleotide as set forth in SEQ ID NO: 14; or
an oligonucleotide as set forth in SEQ ID NO: 15 and an oligonucleotide as set forth in SEQ ID NO: 16.

27-31. (Canceled)

32. A kit, comprising the oligonucleotide according to claims 1.

33. Use of the oligonucleotide according to claim 1 in sequencing.

34. The use according to claim 33, wherein the use in sequencing is high-throughput sequencing, polynucleotide sequencing, forensic science, disease detection, medical diagnosis, precision medicine, companion diagnostics, non-invasive prenatal testing, or early tumor screening.
